(19) United States
(12) Patent Application Publication
(10) Pub. No.: US 2025/0169896 A1
(43) Pub. Date: May 29, 2025

(54) SURGICAL ROBOT, AND POWER SUPPLY SYSTEM, POWER SUPPLY PROTECTION CIRCUIT AND MEDIUM THEREOF

(71) Applicant: Precision Robotics (Hong Kong) Limited, Shatin, N.T. (HK)

(72) Inventors: Ping Lai Benny Lo, Shatin, N.T. (HK); Ka King Wong, Shatin, N.T. (HK)

(21) Appl. No.: 18/698,847

(22) PCT Filed: Aug. 17, 2023
(86) PCT No.: PCT/CN2023/113639

§ 371 Date: Apr. 5, 2024

Related U.S. Application Data
(60) Provisional application No. 63/398,550, filed on Aug. 17, 2022.

(51) Int. Cl. A61B 34/30

(52) U.S. Cl. CPC A61B 34/30

(57) ABSTRACT

The present disclosure provides a surgical robot, a power supply system, a power supply control method, a power supply protection circuit and a medium thereof. The power supply protection circuit includes a main power supply module, a backup power supply module and at least one selection circuit. The selection circuit is configured to determine one of the main power supply module or the backup power supply module as power inputted according to input voltages detected from the main power supply module and the backup power supply module, and output power to supply power to the surgical robot. According to the present disclosure, the control of the automatic selection of power supply is realized by providing the selection circuit for voltage comparison, and then multiple backup power outputs are integrated to the power supply system, so that the surgical robot can work safely and reliably.

### CROSS-REFERENCE TO RELATED APPLICATIONS

This application is a National Stage Application under 35 U.S.C. § 371 of PCT Application No. PCT/CN2023/113639, filed Aug. 17, 2023, which claims priority to U.S. Provisional Patent Application No. 63/398,550, filed Aug. 17, 2022, the entire contents of which are incorporated by reference herein.

### TECHNICAL FIELD

The present disclosure relates the technical field of a surgical robot, and particularly relates to a surgical robot, and a power supply system, a power supply control method, a power supply protection circuit and a medium thereof.

### BACKGROUND

There are high requirements for the stability of power supply in the working process of surgical robots, but various objective factors usually determine that it is difficult to avoid some intermittent power supply stops. The design structure of the surgical robot is often compact, and the application environment space is usually quite limited. Therefore, the existing peripheral backup power module is often difficult to integrate in the surgical robot because of a huge size of the power module, which will affect the normal use of the surgical robot once the surgical robot encounters power failure during its work.

### SUMMARY

A technical problem to be solved by the present disclosure is to provide a power supply protection circuit for a surgical robot for overcoming the above defects in the related art, which includes a main power supply module, a backup power supply module and at least one selection circuit.

The main power supply module and the backup power supply module are located at an input end of the selection circuit and are respectively configured to provide main power and backup power to the selection circuit.

The selection circuit is configured to determine one of the main power supply module or the backup power supply module as power inputted according to input voltages detected from the main power supply module and the backup power supply module, and output power to supply power to the surgical robot.

Preferably, the selection circuit is configured to: select the main power supply module as the power inputted when a first input voltage corresponding to the main power supply module is not less than a second input voltage corresponding to the backup power supply module; and select the backup power supply module as the power inputted when the first input voltage is less than the second input voltage.

Preferably, the backup power supply module is configured to: output power to the selection circuit when the backup power supply module is used as the power inputted, and store power when the main power supply module is used as the power inputted.

Preferably, the power supply protection circuit includes at least two selection circuits with different output voltages.

The present disclosure further provides a power supply system for a surgical robot, which includes the above power supply protection circuit, a power supply interface board and at least one direct current (DC) power control board.

The power supply protection circuit and the DC power control board are respectively electrically connected to the power supply interface board through correspondingly arranged interfaces.

The power supply protection circuit is configured to convert external alternating current (AC) power or Backup power into DC power and output the DC power to the power supply interface board. The DC power control board is configured to obtain the DC power from the power supply interface board and supply power to the surgical robot through its own output interface.

The present disclosure further provides a power supply control method for a surgical robot. The surgical robot includes a power control module for outputting power, and the control method includes the following steps:
reading starting time sequence information of corresponding electrical devices of the surgical robot; and
controlling the power control module to supply power to the electrical devices, according to the starting time sequence information.

Preferably, the power control module includes at least one DC power control board and at least one AC power control board. The electrical devices include a DC electrical device and an AC electrical device. The DC power control board is configured to output DC power to the DC electrical device, and the AC power control board is configured to output AC power to the AC electrical device. The step of controlling the power control module to supply power to the electrical devices in turn includes:
controlling the AC power control board and the DC power control board respectively, to supply power to the DC electrical device and the AC electrical device in turn; where the starting time sequence information is set according to a working parameter of the DC electrical device and the AC electrical device, and the working parameter includes at least one of starting current and rated current.

The present disclosure further provides a power supply control system for a surgical robot, which includes a power control module for outputting power, and the control system includes:
an information reading module, configured to read startup time sequence information of the corresponding electrical devices of the surgical robot; and
a power supply control module, configured to control the power control module to supply power to the electrical devices in turn according to the starting time sequence information.

Preferably, the power control module includes at least one DC power control board and at least one AC power control board. The electrical devices include a DC electrical device and an AC electrical device. The DC power control board is configured to output DC power to the DC electrical device, and the AC power control board is configured to output AC power to the AC electrical device.

The power supply control module is configured to control the AC power control board and the DC power control board respectively, to supply power to the DC electrical device and the AC electrical device in turn. The starting time sequence information is set according to a working parameter of the DC electrical device and the AC electrical device, and the working parameter includes at least one of starting current and rated current.

The present disclosure further provides a computer-readable storage medium, on which a computer program is stored, and when the computer program is executed by the processor, the power supply control method for the surgical robot is realized.

The present disclosure further provides a surgical robot, which includes the power supply system for the surgical robot.

The positive progress effect of the present disclosure is in that: the present disclosure provides a surgical robot, and a power supply system, a power supply control method, a power supply protection circuit and a medium thereof, and the control of the automatic selection of power supply is realized by providing the selection circuit for voltage comparison, and then multiple backup power outputs are integrated to the power supply system, so that the surgical robot can work safely and reliably.

### BRIEF DESCRIPTION OF THE DRAWINGS

FIG. 1 is a schematic diagram of modules of a power supply protection circuit in Embodiment 1 of the present disclosure.

FIG. 2 is a schematic diagram of modules of a power supply protection circuit with multiple selection circuits in Embodiment 1 of the present disclosure.

FIG. 3 is a schematic diagram of modules of a power supply system in Embodiment 2 of the present disclosure.

FIG. 4 is a schematic diagram of the arrangement of DC power control modules of the power supply system in Embodiment 2 of the present disclosure.

FIG. 5 is a flowchart of a power supply control method for a surgical robot in Embodiment 3 of the present disclosure.

FIG. 6 is a schematic diagram of modules of a power supply control system for a surgical robot in Embodiment 4 of the present disclosure.

### DETAILED DESCRIPTION

In order to explain the technical solution of the embodiments of this specification more clearly, the accompanying drawings needed in the description of the embodiments will be briefly introduced below. Obviously, the accompanying drawings in the following description are only some examples or embodiments of this specification. For those skilled in the art, this specification can be applied to other similar situations according to these accompanying drawings without creative work. Unless it is obvious from the linguistic context or otherwise stated, the same reference signs in the accompanying drawings represent the same structure or operation.

As shown in this specification, the words “a”, “an”, “one” and/or “the” do not refer to the singular, but may also include the plural. Generally, the terms “include” and “contain” only imply the inclusion of clearly identified steps and elements, and these steps and elements do not constitute an exclusive list, and a method or device may also contain other steps or elements.

### Embodiment 1

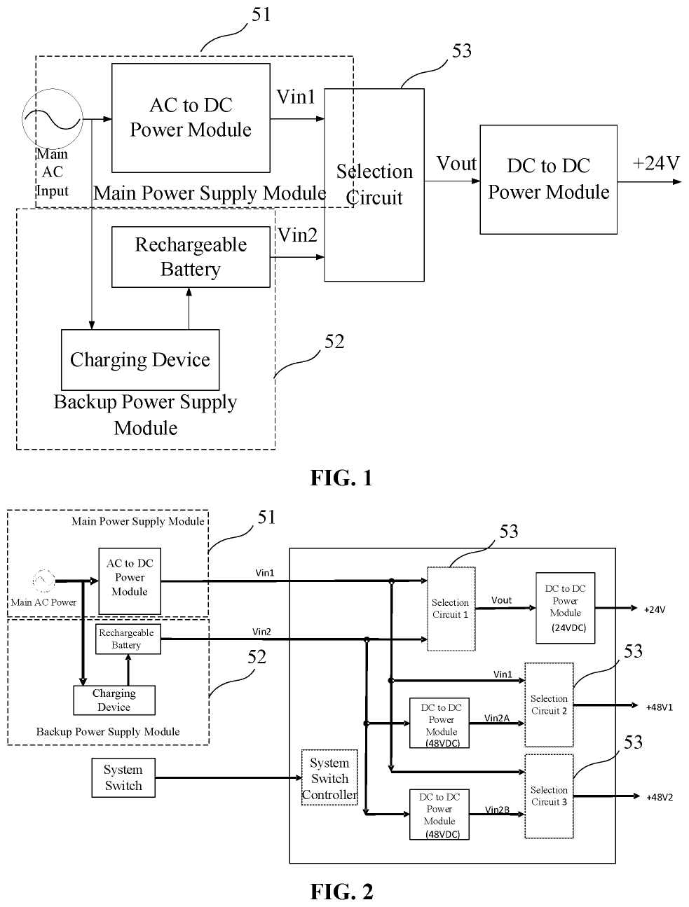

As shown in FIG. 1, this embodiment specifically provides a power supply protection circuit for a surgical robot, including a main power supply module 51, a backup power supply module 52 and at least one selection circuit 53.

The main power supply module 51 and the backup power supply module 52 are located at an input end of the selection circuit 53, and are respectively configured to provide main power and backup power to the selection circuit 53. It can be understood that in this embodiment, the AC input is used as the main power supply module 51, and a plurality of DC to DC transformers are provided in the power supply protection circuit, the input ends of which are electrically connected with the rechargeable battery, and the output ends of which are electrically connected with the corresponding selection circuit 53, and the main AC input can be converted into DC power for use.

The selection circuit 53 is configured to determine one of the main power supply module 51 and the backup power supply module 52 as power inputted according to input voltages detected from the main power supply module 51 and the backup power supply module 52, and output power to supply power to the surgical robot.

As a preferred embodiment, the selection circuit 53 is configured to: select the main power supply module 51 as the power inputted when a first input voltage corresponding to the main power supply module 51 is not less than a preset voltage threshold; and select the backup power supply module 52 as the power inputted when the first input voltage is less than the preset voltage threshold. This embodiment realizes automatic control and selection of power supply through numerical comparison and logical judgment of the first input voltage and the second input voltage, and ensures the safe and normal power supply of the surgical robot. In this embodiment, by logically comparing and controlling the input voltages corresponding to the main power supply module 51 and the backup power supply module 52, it is possible to reasonably define the currently suitable input power to ensure power supply.

As a preferred embodiment, the backup power supply module 52 is configured to output power to the selection circuit when it is used as power inputted, and store power when the main power supply module 51 is used as power inputted. The backup power supply module 52 includes a charging device and a rechargeable battery which are electrically connected with each other. The charging device is configured to store power in the rechargeable battery when the main power supply module 51 is used as the power inputted. In this embodiment, by using the properties of the charging device and the rechargeable batteries in the power supply module 52, synchronous power storage can be performed when the system is in normal operation, that is, power is supplied by the main power supply module 51, so that the backup power can be kept sufficient, and the backup power can be provided in time when necessary.

Preferably, the power supply protection circuit includes at least two selection circuits 53 with different output voltages. As shown in FIG. 2, multiple selection circuits can be provided to perform logic processing and output power supply synchronously, so as to form more judgment combinations and output modes, thus meeting the requirements of different electrical device.

The power supply protection circuit for the surgical robot in this embodiment realizes the control of the automatic selection of power supply by reasonably providing the selection circuit for voltage comparison, so that on the one hand, the safe and normal use of the surgical robot can be ensured, and on the other hand, a battery of the backup power can be kept charged without being exhausted under the condition of normal power supply.

### Embodiment 2

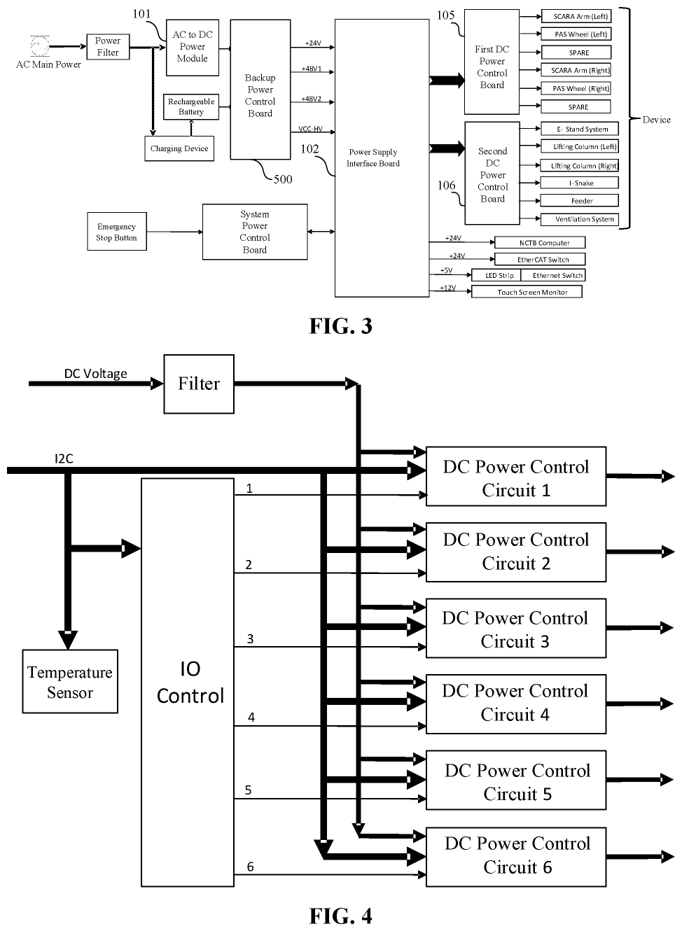

As shown in FIGS. 3-4, this embodiment specifically provides a power supply system for a surgical robot, including an AC to DC power module 101, a power supply interface board 102 and at least one DC power control board. The AC to DC power module 101 and the DC power control board are respectively electrically connected to the power supply interface board 102 through correspondingly arranged interfaces. The AC to DC power module 101 is configured to convert obtained external AC power into DC power and output the DC power to the power supply interface board 102. Preferably, the AC to DC power module may be a power supply unit (PSU). The DC power control board is configured to obtain the DC power from the power supply interface board 102 and supply power to the surgical robot through its own output interface. Those skilled in the art can understand that the power supply interface board 102 may be a field programmable gate array (FPGA) component. The DC power control board may be electrically connected to the power supply interface board 102 through a corresponding interface, or may be connected by other means.

Preferably, the power supply interface board 102 may also be provided with several interfaces for directly outputting power supply, such as being connected to a display device, etc. The layout of the interfaces may be uniformly designed in combination with the number of interfaces of the DC power control board and the size of the power supply interface board 102, for example, the interface for directly outputting power supply is arranged on the other side of the DC power control board.

As a preferred embodiment, the power supply system for the surgical robot further includes a DC to DC power module 103. The DC to DC power module 103 is electrically connected to the AC to DC power module 101 and the power supply interface board 102, respectively. The AC to DC power module 101 directly outputs first DC power to the power supply interface board 102, and further outputs second DC power to the power supply interface board 102 through the DC to DC power module 103. The power supply interface board 102 provides 48V DC power or 24V DC power to the corresponding DC power control board, respectively. The first DC power and the second DC power in this embodiment are described by taking 48V DC power and 24V DC power as an example respectively, and those skilled in the art can understand that they do not constitute any restrictions on the present disclosure.

In this embodiment, by providing the DC to DC power module 103, the power supply system not only provides a DC input directly from the AC to DC power module to the power supply interface board, but also provides another DC input in parallel, thus enriching the output parameter selection of the power supply interface board. At the same time, the DC to DC power module 103 itself can also directly supply power to the appropriate devices simultaneously, which is beneficial to the adaptability of the intraoperative environment and is better compatible with some scenarios with many operating room devices.

As a preferred embodiment, the power supply system for the surgical robot further includes a first DC power control board 105 and a second DC power control board 106. The first DC power control board 105 and the second DC power control board 106 respectively obtain the 48V DC power and the 24V DC power from the power supply interface board 102 through correspondingly arranged interfaces, and provide the 48V DC power and the 24V DC power to the surgical robot through their own output interfaces.

In this embodiment, two DC power control boards corresponding to different output currents are provided to meet the complicated power supply requirements of the surgical robot. Of course, as mentioned above, the 48V and 24V DC power are just examples herein. According to the needs of the actual environment and different devices, other DC power control boards may be added or output parameters of their corresponding power may be changed.

As a preferred embodiment, the first DC power control board 105 and the second DC power control board 106 respectively include a current sensor, a voltage sensor, a temperature sensor and a circuit protection component arranged corresponding to the output interfaces. The circuit protection component may be a resettable fuse, and at the same time, a plurality of groups of the same or similar DC power control boards may be provided to form a DC output that meets the needs. As shown in FIG. 4, a plurality of DC power control modules may be set for parallel output, and the DC power control module may be the DC power control board mentioned above. In this embodiment, the DC power control board is provided with a monitoring module such as a voltage sensor and a circuit protection component such as a resettable fuse, which can form an effective monitoring and repair mechanism for the DC power control board which is one of the core components of the power supply system, ensuring that the DC power control board can provide a stable DC power output and provide safe and stable output supply for the electrical device.

As a preferred embodiment, the power supply system for the surgical robot further includes an AC power control board. The external AC power is inputted to the AC power control board, so that the power supply system outputs AC power. At the same time, the external AC power is inputted to the AC to DC power module 101, so that the power supply system outputs DC power. Preferably, the power supply system for the surgical robot further includes a power filter 104, which is configured to carry out an electromagnetic finishing treatment on the external AC power. The power filter is electrically connected to the AC power control board and the AC to DC power module 101 respectively. By providing the power filter 104, the power inputted is stably controlled. While the AC power control board 107 performs conversion through the AC to DC power module 101, it directly outputs AC power, which can make the output form of the whole power supply system more abundant and reasonable, and meet the requirements of different electrical devices.

Based on the power supply protection circuit in Embodiment 1, the power supply system in this embodiment can supply power to the surgical robot continuously and stably through reasonable judgment logic when the normal power supply encounters an accident, so as to ensure the normal operation of the surgical robot.

The AC power input in the power supply system is realized by the power supply system for the surgical robot in this embodiment based on providing the AC to DC power module, the power supply interface board and the DC power module, so that the DC power may be directly obtained by all related system components, and there is no need to set an inverter in the whole power supply system, thus simplifying the structure of the power on the premise of ensuring the power supply requirements, avoiding the circuit loss caused thereby. At the same time, based on the power supply protection circuit, the power supply system for the surgical robot can continuously and stably supply power to the surgical robot in the scene of unexpected power failure.

### Embodiment 3

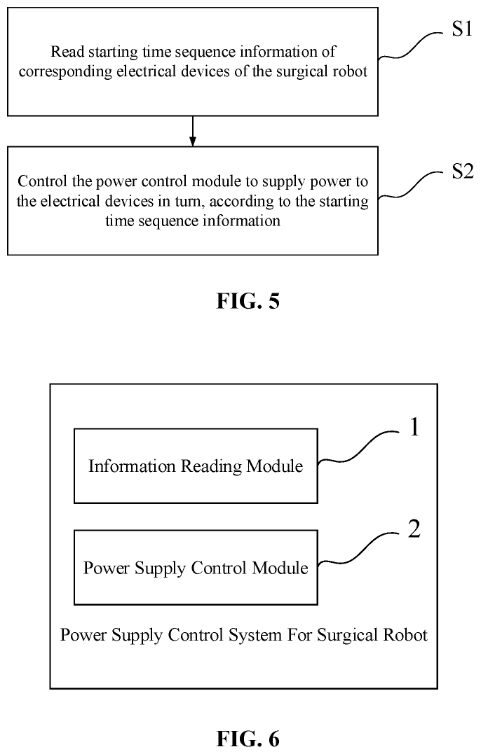

As shown in FIG. 5, this embodiment specifically provides a power supply control method for a surgical robot. The surgical robot includes a power control module for outputting power, and the control method includes:

S1, reading starting time sequence information of corresponding electrical devices of the surgical robot; and

S2, controlling the power control module to supply power to the electrical devices in turn, according to the starting time sequence information.

The surgical robot may include a body and different external electrical devices. The power supply control method in this embodiment supplies power to different devices based on the power control module of the surgical robot. In a preferred embodiment, the power control module outputs DC power for supplying power by converting and controlling the input power.

In step S1, corresponding starting time sequence information is obtained for different electrical devices, and the time sequence information may be expressed by relative time (such as the startup sequence of several devices), programmed time (such as turning on a device when it reaches a certain moment) or their combination. On this basis, in step S2, the power control module is controlled to power on each electrical device in turn according to the starting time sequence, so as to realize power consumption peak shifting and avoid the corresponding over-current phenomenon. Of course, several electrical devices involved in the starting time sequence may be started at the same time, and there is no restriction that they must have a sequence, as long as they meet the requirements of over-current protection. Preferably, when the surgical robot is started, step S1 is automatically executed to start the detection and control of power supply.

As a preferred embodiment, the power control module includes at least one DC power control board and at least one AC power control board. The electrical devices include a DC electrical device and an AC electrical device. The DC power control board is configured to output DC power to a DC electrical device, and the AC power control board is configured to output AC power to an AC electrical device. Step S2 includes:
controlling the AC power control board and the DC power control board to supply power to the DC electrical device and the AC electrical device in turn, respectively; where, the starting time sequence information is set according to a working parameter of the DC electrical device and the AC electrical device, and the working parameter includes at least one of starting current and rated current.

When the power supply system is provided with the DC power output and the AC power output respectively, the types of the above-mentioned power outputs are distinguished and corresponding control is carried out in combination with the types of corresponding electrical device, so that the starting time sequence of the electrical device may be determined according to the working parameter such as the starting current and rated current, so as to ensure a stable output under the condition that the power supply system corresponds to a variety of different devices.

The power supply control method for the surgical robot in this embodiment is based on the analysis of different electrical components of the surgical robot, and the corresponding electrical control logic is reasonably designed, so that the components of the surgical robot system can obtain independent DC power supply. By controlling the power-on time of the electrical components, the current surge can be effectively prevented when the electrical components are powered on, and the surgical robot can effectively work safely and stably, which has the significance of industry promotion.

### Embodiment 4

As shown in FIG. 6, this embodiment specifically provides a power supply control system for a surgical robot. The surgical robot includes a power control module for outputting power, and the control system includes:
an information reading module 1, configured to read starting time sequence information of corresponding electrical devices of the surgical robot; and
a power supply control module 2, configured to control the power control module to supply power to the electrical devices in turn according to the starting time sequence information.

The surgical robot may include a body and different external electrical devices. The power supply control method in this embodiment supplies power to different devices based on the power control module of the surgical robot. In a preferred embodiment, the power control module outputs DC power for supplying power by converting and controlling the input power.

The information reading module 1 obtains corresponding starting time sequence information for different electrical devices, and the time sequence information may be expressed by relative time (such as the startup sequence of several devices), programmed time (such as turning on a device when it reaches a certain moment) or their combination. On this basis, the power supply control module 2 controls the power control module to power on each electrical device in turn according to the starting time sequence, so as to realize power consumption peak shifting and avoid a corresponding over-current phenomenon. Of course, several electrical devices involved in the starting time sequence may be started at the same time, and there is no restriction that they must have a sequence, as long as they meet the requirements of over-current protection. Preferably, when the surgical robot is started, the information reading module 1 is automatically called to start the detection and control of power supply.

As a preferred embodiment, the power control module includes at least one DC power control board and at least one AC power control board. The electrical devices include a DC electrical device and an AC electrical device. The DC power control board is configured to output DC power to the DC electrical device, and the AC power control board is configured to output AC power to the AC electrical device. The power supply control module 2 is specifically configured to:
control the AC power control board and the DC power control board respectively, to supply power to the DC electrical device and the AC electrical device in turn; and the starting time sequence information is set according to a working parameter of the DC electrical device and the AC electrical device, and the working parameter includes at least one of starting current and rated current.

When the power supply system is provided with a DC power output and an AC power output respectively, this embodiment distinguishes types of the above power supply outputs and carries out corresponding control in combination with types of the corresponding electrical devices. Specifically, the starting time sequence of the electrical devices may be determined according to the working parameter such as the starting current and rated current, so as to ensure stable output when the power supply system corresponds to a variety of different devices.

The power supply control system for the surgical robot in this embodiment rationally designs the corresponding power control logic based on the analysis of different electrical components of the surgical robot, so that the components of the surgical robot system can obtain independent DC power supply. By controlling the power-on time of the electrical components, the current surge can be effectively prevented when the electrical components are powered on, so that the surgical robot can effectively work safely and stably, and the present disclosure has the significance of industry promotion.

### Embodiment 5

This embodiment further provides a computer-readable storage medium, on which a computer program is stored, and when executed by a processor, the computer program realizes the steps in the power supply control method for the surgical robot of the above embodiment. The readable storage medium may be a portable disk, a hard disk, a random-access memory, a read-only memory, an erasable programmable read-only memory, an optical storage device, a magnetic storage device or any suitable combination of the above.

In a possible embodiment, the present disclosure may also be realized in the form of a program product, which includes a program code, and when the program product is run on a terminal device, the program code is used to make the terminal device execute the steps in the power supply control method for the surgical robot as described above. The program code for executing the present disclosure may be written in any combination of one or more programming languages, and the program may be completely executed on a user device, partially executed on the user device, executed as an independent software package, partially executed on the user device and partially executed on a remote device, or completely executed on the remote device.

### Embodiment 6

This embodiment provides a surgical robot, which is realized based on the power supply system in Embodiment 2 and can work continuously and stably in the scene of unexpected power failure. It can be understood that the surgical robot can be a surgical robot platform including a body device and an auxiliary device.

Although specific embodiments of the present disclosure have been described above, those skilled in the art should understand that these are merely illustrative, and the scope of protection of the present disclosure is defined by the appended claims. Those skilled in the art may make many changes or modifications to these embodiments without departing from the principle and substance of the present disclosure, but these changes and modifications all fall within the protection scope of the present disclosure.

1. A power supply protection circuit for a surgical robot, comprising a main power supply module, a backup power supply module and at least one selection circuit, wherein,
the main power supply module and the backup power supply module are located at an input end of the selection circuit and are respectively configured to provide main power and backup power to the selection circuit; and
the selection circuit is configured to determine one of the main power supply module and the backup power supply module as power inputted according to input voltages detected from the main power supply module and the backup power supply module, and output power to supply power to the surgical robot.

2. The power supply protection circuit for the surgical robot according to claim 1, wherein the selection circuit is configured to: select the main power supply module as the power inputted in response to that a first input voltage corresponding to the main power supply module is not less than a second input voltage corresponding to the backup power supply module; and select the backup power supply module as the power inputted in response to that the first input voltage is less than the second input voltage.

3. The power supply protection circuit for the surgical robot according to claim 2, wherein the backup power supply module is configured to: output power to the selection circuit in response to that the backup power supply module is used as the power inputted, and store power in response to that the main power supply module is used as the power inputted.

4. The power supply protection circuit for the surgical robot according to claim 3, wherein the power supply protection circuit comprises at least two selection circuits with different output voltages.

5. A power supply system for a surgical robot, comprising the power supply protection circuit according to claim 1, a power supply interface board and at least one direct current (DC) power control board, wherein,
the power supply protection circuit and the DC power control board are respectively electrically connected to the power supply interface board through correspondingly arranged interfaces; and
the power supply protection circuit is configured to convert external alternating current (AC) power or backup power into DC power and output the DC power to the power supply interface board; and the DC power control board is configured to obtain the DC power from the power supply interface board and supply power to the surgical robot through its own output interface.

6. A power supply control method for a surgical robot, wherein the surgical robot comprises a power control module for outputting power, and the control method comprises:
reading starting time sequence information of corresponding electrical devices of the surgical robot;
controlling the power control module to supply power to the electrical devices, according to the starting time sequence information.

7. The power supply control method for the surgical robot according to claim 6, wherein the power control module comprises at least one direct current (DC) power control board and at least one alternating current (AC) power control board; the electrical devices comprise a DC electrical device and an AC electrical device; the DC power control board is configured to output DC power to the DC electrical device, and the AC power control board is configured to output AC power to the AC electrical device; and the step of controlling the power control module to supply power to the electrical devices in turn comprises:
controlling the AC power control board and the DC power control board respectively, to supply power to the DC electrical device and the AC electrical device in turn; wherein the starting time sequence information is set according to a working parameter of the DC electrical device and the AC electrical device, and the working parameter comprises at least one of starting current and rated current.

8. (canceled)

9. (canceled)

10. (canceled)
